(19) United States
(12) Patent Application Publication
(10) Pub. No.: US 2002/0068290 A1
(43) Pub. Date: Jun. 6, 2002

(54) Topoisomerase activated oligonucleotide adaptors and uses therefor

(76) Inventor: Timur Yarovinsky, Coralville, IA (US)

Correspondence Address: FOLEY, HOAG & ELIOT, LLP, PATENT GROUP, ONE POST OFFICE SQUARE, BOSTON, MA, 02109, US

(21) Appl. No.: 09/871,607

(22) Filed: May 31, 2001

Related U.S. Application Data
(60) Provisional application No. 60/208,662, filed on May 31, 2000.

(51) Int. Cl. C12Q 1/68; C07H 21/04; C12P 19/34

(52) U.S. Cl. 435/6; 435/91.2; 536/23.2

(57) ABSTRACT

The invention provides methods and compositions for the rapid joining of a target nucleic acid sequence with a topoisomerase activated adaptor sequence that provides a specific function to the target.

### CROSS-REFERENCE TO RELATED APPLICATIONS

This application claims the benefit of U.S. Provisional Application No. 60/208,662, filed May 31, 2000, the contents of which are specifically incorporated herein.

### 1. BACKGROUND OF THE INVENTION

The ability to clone nucleic acid sequences that encode specific genetic functions followed the elucidation of the chemical structure of DNA and the later discovery of enzymes that cleave at specific nucleotide sequences (i.e. the restriction endonucleases) and that catalyze the joining of nucleic acid fragments with compatible ends (i.e. the DNA ligases). More recently it was discovered that vaccinia DNA topoisomerase I, which functions in vivo to relax the supercoiled chromosomal and episomal DNA, may be used to both cleave at a specific nucleotide sequence and to subsequently catalyze the joining of the cleaved sequence to a nucleic acid fragment with a compatible end. Vaccinia topoisomerase I cleaves at the 3′end of the consensus five base sequence element (C/T)CCTT. In the cleavage reaction, bond energy is conserved via the formation of a covalent adduct between the 3′ phosphate of the incised strand and a tyrosyl residue (Tyr-274) present in the catalytic site of the topo I (Shuman et al. (1989) Proc Natl Acad Sci USA 86: 9793-7). If the nucleic acid associated with the free 5′-end created by the topo I-catalyzed cleavage event is allowed to diffuse away, another nucleic acid fragment with a compatible end including a free 5′ hydroxyl tail may be joined to the topo I-activated fragment.

Heyman et al. (1999) Genome Research 9: 383-92 describes a multi-step method for the preparation of a topoisomerase activated cloning vector using adaptor sequences compatible with unique Hind III site in the vector. The method requires the cloning of an adaptor sequence consisting of two single stranded oligonucleotides (i.e. TOPO-H and TOPO-4) to the Hind III site in the vector using a DNA ligase. This is followed by the addition of Vaccinia topoisomerase and a third oligonucleotide which is complementary to the 3′ end of the Vaccinia topoisomerase recognition sequence present in TOPO-H (i.e. TOPO-5). The Vaccinia topoisomerase cleaves after the double strand CCCTT recognition sequence present in the adapor and the TOPO-5 oligonucleotide then dissociates leaving 3′T-overhangs that are covalently associated with topoisomerase I on the vector. This vector can then be used in cloning a target nucleic acid sequence.

U.S. Pat. No. 5,766,891 describes a method for the molecular cloning of DNA by PCR-mediated introduction of a topoisomerase cleavage site into a target DNA sequence. The resulting amplification product is reacted with topoisomerase and the activated sequence is then directly cloned into a compatible vector.

### 2. SUMMARY OF THE INVENTION

The present invention provides compositions and methods for the rapid joining of a target nucleic acid sequence having a one-base 3′ overhang to an activated oligonucleotide adaptor sequence. The adaptor sequence may be customized to provide a particular function to the target nucleic acid sequence such as: a prokaryotic promoter sequence, a eukaryotic promoter sequence, a viral promoter sequence, a mutational sequence, a single-stranded overhang sequence, a nucleic acid sequence tag, a polypeptide sequence tag, or a chemical group such as a radioactive label or a chemical ligand. Generally, the target nucleic acid sequence is a double stranded nucleic acid molecule possessing a terminal 3′-dAMP residue, such as is produced by various thermophilic polymerases during PCR amplification, and a free 5′-hydroxyl group, such as is provided by the oligonucleotide primer ends of a PCR amplification product or by phosphatase treatment of a restriction endonuclease cleavage product. The invention may also be adapted to target nucleic acid sequence possessing a one-base overhang other than a 3 ′-dAMP overhang. In certain instances, the invention may be adapted to target nucleic acid sequences which are blunt-ended. The adaptors may be generated by the hybridization of two synthetic oligonucleotides followed by activation with a topoisomerase type I enzymatic activity such as provided by vaccinia virus topoisomerase I. The topoisomerase activated adaptors are then incubated with the target nucleic acid sequence to allow the topoisomerase-catalyzed joining of the adaptor sequence to the target sequence. The joined product may be used directly as desired. Preferred uses of the joined product will be dictated by the nature of the particular functional sequence that was included in the customized adaptor sequence utilized.

### 3. BRIEF DESCRIPTION OF THE FIGURES

FIG. 1 illustrates the formation of topoisomerase-activated adaptors from synthetic oligonucleotides.

FIG. 2 depicts the modification of PCR products with topoisomerase-activated adaptors.

FIG. 3 shows the results of in situ hybridization of monkey retina with a cRNA probe generated from the T7 adaptor-cDNA PCR product.

### 4. DETAILED DESCRIPTION OF THE INVENTION

4.1. General

In general, the invention provides reagents and methods for the joining of a topoisomerase activated adaptor encoding a function with a nucleic acid target or acceptor molecule. The adaptor sequence is designed to include a topoisomerase recognition sequence which, upon incubation with topoisomerase, results in cleavage and covalent activation. The function provided by the activated adaptor may be any of a number of encoded functionalities, such as a promoter sequence, or functional groups, such as an affinity tag.

4.2. Definitions

For convenience, the meaning of certain terms and phrases employed in the specification, examples, and appended claims are provided below.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

“Biological activity” or “bioactivity” or “activity” or “biological function”, which are used interchangeably, for the purposes herein means an effector or antigenic function that is directly or indirectly performed by a polypeptide (whether in its native or denatured conformation), or by any subsequence thereof. Biological activities include binding to a target peptide, e.g., a receptor.

The term “biomarker” refers a biological molecule, e.g., a nucleic acid, peptide, hormone, etc., whose presence or concentration can be detected and correlated with a known condition, such as a disease state.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding a first subject polypeptides with a second amino acid sequence defining a domain (e.g. polypeptide portion) foreign to and not substantially homologous with any domain of the first polypeptide. A chimeric polypeptide may present a foreign domain which is found (albeit in a different polypeptide) in an organism which also expresses the first polypeptide, or it may be an “interspecies”, “intergenic”, etc. fusion of polypeptide structures expressed by different kinds of organisms. In general, a fusion polypeptide can be represented by the general formula X-polypep.-Y, wherein polypep. represents a portion or all of a first subject polypeptide sequence, and X and Y are independently absent or represent amino acid sequences which are not related to the first polypeptide sequence in an organism, including naturally occurring mutants.

The term “complementary” and “compatible” is used herein to describe the capacity of a pair of single-stranded terminal sequences to anneal to each other via base pairing (e.g. A-T or G-C).

A “delivery complex” shall mean a targeting means (e.g. a molecule that results in higher affinity binding of a gene, protein, polypeptide or peptide to a target cell surface and/or increased cellular or nuclear uptake by a target cell). Examples of targeting means include: sterols (e.g. cholesterol), lipids (e.g. a cationic lipid, virosome or liposome), viruses (e.g. adenovirus, adeno-associated virus, and retrovirus) or target cell specific binding agents (e.g. ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As used herein, the term “enhancer” refers to a DNA sequence, which, without regard to its position or orientation in the DNA, increases the amount of RNA synthesized from an associated promoter. Enhancers are typically found in association with eukaryotic or viral promoters and frequently confer tissue-specific and/or developmental-specific expression of the linked promoter.

The term “equivalent” is understood to include nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of a specified nucleic acids, due to the degeneracy of the genetic code.

As used herein, the term “handle” is used to describe a chemical or biochemical modification to a nucleotide residue within an oligonuclotide or a nucleic acid component. A handle provides a site for covalent or non-covalent attachment of a biological or chemical molecule(s) to a nucleic acid, such as an adaptor/acceptor nucleic acid conjugate.

The term “hapten” refers to a small molecule that acts as an antigen when linked to a protein.

As used herein, the term “genetic element” describes a sequence of nucleotides including those which encode a regulatory region, involved in modulating or producing biological activity or responses or which provides a specific signal involved in a molecular mechanism or biological activity. For example, a prokaryotic gene may be comprised of several genetic elements including a promoter, a protein coding region, a Shine-Delgamo sequence, and translational and transcriptional initiators and terminators.

As used herein, the term “functionality” describes the normal characteristic utility or utilities of a synthetic construct, a gene, a gene fragment, or one or more genetic elements.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with a specified sequence of the present invention.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the subject gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.). In preferred embodiments, the “knock-out” gene locus corresponding to the modified endogenous gene no longer encodes a functional polypeptide activity and is said to be a “null” allele.

A “knock-out construct” refers to a nucleic acid sequence that can be used to decrease or suppress expression of a protein encoded by endogenous DNA sequences in a cell. In a simple example, the knock-out construct is comprised of a gene with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: target gene 5′/neo/target gene 3′, where target gene 5′ and target gene 3′, refer to genomic or cDNA sequences which are, respectively, upstream and downstream relative to a portion of the target gene and where neo refers to a neomycin resistance gene. In another knock-out construct, a second selectable marker is added in a flanking position so that the gene can be represented as: target gene 5′/neo/target gene 3′/TK, where TK is a thymidine kinase gene which can be added to either the target gene 5′ or the target gene 3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

The term “linkage” refers to a physical connection, preferably covalent coupling, between two or more nucleic acid components, e.g. catalyzed by an enzyme such as a ligase.

The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant IBR genes is present and/or expressed or disrupted in some tissues but not others.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “nucleotide sequence complementary to the nucleotide sequence set forth in SEQ ID No. x” refers to the nucleotide sequence of the complementary strand of a nucleic acid strand having SEQ ID No. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID No. x refers to the complementary strand of the strand having SEQ ID No. x or to any nucleic acid having the nucleotide sequence of the complementary strand of SEQ ID No. x. When referring to a single stranded nucleic acid having the nucleotide sequence SEQ ID No. x, the complement of this nucleic acid is a nucleic acid having a nucleotide sequence which is complementary to that of SEQ ID No. x. The nucleotide sequences and complementary sequences thereof are always given in the 5′ to 3′ direction.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterrnan algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ).

Preferred nucleic acids have a sequence at least 70%, and more preferably 80% identical and more preferably 90% and even more preferably at least 95% identical to an nucleic acid sequence of a specified sequence shown. Nucleic acids at least 90%, more preferably 95%, and most preferably at least about 98-99% identical with a specified nucleic sequence represented are of course also within the scope of the invention. In preferred embodiments, the nucleic acid is mammalian. In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” refers to a DNA sequence which is recognized by an RNA polymerase and which directs initiation of transcription at a nearby downstream site. As used herein “promoter” refers to viral, phage, prokaryotic or eukarytoic transcriptional control sequences. Generally, term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The term “recombinant protein” refers to a polypeptide of the present invention which is produced by recombinant DNA techniques, wherein generally, DNA encoding an IBR polypeptide is inserted into a suitable expression vector which is in turn used to transform a host cell to produce the heterologous protein. Moreover, the phrase “derived from”, with respect to a recombinant IBR gene, is meant to include within the meaning of “recombinant protein” those proteins having an amino acid sequence of a native IBR polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate an IBR bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably an IBR gene.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of one of the IBR genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of IBR polypeptide.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of an IBR polypeptide or, in the case of anti-sense expression from the transferred gene, the expression of a naturally-occurring form of the IBR polypeptide is disrupted.

As used herein, the term “transgene” means a nucleic acid sequence (encoding, e.g., one of the IBR polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the IBR polypeptides, e.g. either agonistic or antagonistic forms. However, transgenic animals in which the recombinant target gene is silent are also contemplated, as for example, the FLP or CRE recombinase dependent constructs described below. Moreover, “transgenic animal” also includes those recombinant animals in which gene disruption of one or more IBR genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto, for example linear vectors. Examples of linear vectors include various viral genomes as well as yeast artificial chromosomes (YACs) and mammalian artificial chromosomes (see e.g. Grimes and Cooke (1998) Hum Mol Genet, 7: 1635-40; and Vos (1998) Curr Opin Genet Dev, 8: 351-9).

4.3. Oligonucleotide Adaptors

In general, the invention provides oligonucleotide adaptor sequences which comprise: (1) a topoisomerase recognition/cleavage sequence; and (2) a functional group or encoded functionality. Topoisomerase recognition and cleavage sequences are discussed below in section 4.4. In addition, the oligonucleotides of the invention may be composed of conventional deoxyribonucleotide or ribonucleotide units or modified synthetic oligonucleotide structures which are known in the art and discussed further below.

Functional group which may be incorporated into the oligonucleotide adaptors of the invention include biotin, fluorescent tags, haptens, affinity tags, and lipophilic membrane targeting groups. Such conjugate groups may be coupled to the oligonucleotides either through sites present naturally in nucleic acids or through some other reactive linker group introduced specifically for the purpose. The naturally occurring groups that can be used include amino groups on the bases, hydroxyl groups on the sugars, and terminal and internal phosphate groups. Linker groups attached to the oligonucleotide for derivation are most commonly primary amines, thiols, or aldehydes, but other types of chemical linker groups are also possible. In some instances, the linker group is attached to the oligonucleotide by a spacer arm either to facilitate coupling or to distance the conjugate group from the oligonucleotide. Furthermore, either the conjugate group or the linker may be introduced at any one of three stages during oligonucleotide synthesis as follows: by attachment to a nucleotide before incorporation into the growing chain; by attachment to the oligonucleotide after synthesis by deblocking; by chemical attachment within the synthetic oligonucleotide between nucleotide units. The chemistry for effecting such attachments is well known (see Goodchild (1990) Bioconjugate Chemistry 1: 165-187 for review). Examples of preferred functional groups include: fluorescent dyes including fluoresceins, tetramethylrhodamine, Texas red, pyrene, bimane, mansyl, dansyl, proflavine, eosin, naphtalene derivatives and coumarin derivatives; intercalating agents including acridine, oxazolopyridocarbazole, anthraquinone, phenanthridine and phenazine; proteins including peroxidases, antibodies (e.g. IgG), alkaline phosphatases, polylysine and nucleases; cross-linking agents such as alkylating agents, azidobenzenes, psoralen, iodoacetamide, azidoproflavin, azidouracil, and platinum; chain-cleaving agents including EDTA/Fe⁺², phnanthroline/Cu⁺², and porphyrin/Fe⁺²; and other conjugatable functional groups including biotin, solid support matrixes, dinitrophenyl, trinitrophenyl, proxyl spin-label, fluorene, isoluminol, digoxigenin, puromycin, DTPA and other chelating agents, phopholipid, and cholesterol.

For example, the synthesis of biotinylated nucleotides is well known in the art and was first described by Langer et al. ((1981) Proc Natl Acad Sci USA 78: 6633-37). The water 35 soluble biotin group may be covalently attached to the C5 position of the pyrimidine ring via an allylamine linker arm. Biotinylated nucleic acid molecules can be prepared from biotin-NHS (N-hydroxy-succinimide) using techniques well known in the art (e.g. biotinylation kit, Pierce Chemicals, Rockford, Ill.).

Functionalities encoded by the oligonucleotide adaptor sequences of the invention include promoter sequences, enhancer sequences, transcription initiation sequences, transcription termination sequences, polyadenylation signals, intronic sequences, translation initiation sequences, epitope tag sequences, integration-promoting factor sequences, an mRNA stability-regulating sequence, restriction endonuclease recognition/cleavage sequences, synthetic multiple cloning site sequences, cellular localization encoding sequences, and sites for the covalent or noncovalent attachment of a biological or chemical functional group (as described above). For example, exemplary promoter sequences include phage, viral, prokaryotic and eukaryotic promoter elements. Preferred prokaryotic phage promoter elements include lambda phage promoters (e.g. P_{RM} and P_{R}), T7 phage promoter sequences (e.g. TAATACGACTCACTATA), T3 phage promoter sequences (e.g. TTATTAACCCTCACTAAAGGGAAG), and SP6 phage promoter sequences (e.g. ATTTAGGTGACACTATAGAATAC). Preferred prokaryotic promoter elements include those carrying optimal −35 and −10 (Pribnow box) sequences for transcription by a prokaryotic (e.g. E. coli) RNA polymerase. In addition, some prokaryotic promoters contain overlapping binding sites for regulatory repressors (e.g. the Lac promoter and the synthetic TAC promoter, which contain overlapping binding sites for lac repressor thereby conferring inducibility by the substrate homolog IPTG). Prokaryotic genes from which suitable promoters sequences may be obtained include the E. coli lac, ara and trp genes. Preferred eukaryotic promoter sequences include eukaryotic viral gene promoters such as those of the SV40 promoter, the herpes simplex thymidine kinase promoter, as well as any of the various retroviral LTR promoter elements (e.g. the MMTV LTR).

It is further understood that the invention is not limited to oligonucleotide adaptor compositions comprised of conventional deoxyribonucleotide or ribonucleotide units.

Modifications to the oligonucleotide have been frequently employed for use in antisense inhibition where it is necessary for oligonucleotides to remain stable in cell culture or other biological environments and also where the ability to cross lipophilic cell membranes is critical. Changes may be made at the bases, the sugars, the ends of the chain, or at the phosphate groups of the backbone. Alterations of the bases or sugars must be designed so as to avoid disrupting hydrogen bonding critical to essential oligonucleotide base pairing interactions. Modification to the ends and backbone of the molecule are generally easier to effect and these sites provide a convenient point for attachment of the functional groups discussed above. Furthermore, as the ends of the oligonucleotide are the site of action of most nucleases and also carry charges that inhibit cellular uptake, this presents the most direct approach to improvement in these areas. Chemically modified phosphate backbones for use in the oligonucleotides of the invention include methylphosphonates, phosphotriesters, phosphorothioates and phosphoramidates (see Goodchild (1990) Bioconjugate Chemistry 1: 165-187 for review). The selection of appropriate phosphate backbone modifications for use in the invention will be directed by the intended use of the adaptor or adaptor-target nucleic acid topoisomerase ligation product. Considerations include required chemical and biological stability and lipophilic properties. Advantages of particular modified phosphate groups are well known in the art and have been reviewed in detail (see Goodchild (1990) Bioconjugate Chemistry 1: 165-187).

4.4. Topoisomerase I and Topoisomerase Activation

The invention can be used in conjunction with numerous naturally occurring and genetically engineered topoisomerase I activities. The eukaryotic topoisomerase IB family (see Wang (1996) 65:635-92) includes topoisomerase I and the topoisomerases encoded by vaccinia and other cytoplasmic poxviruses. These enzymes catalyze DNA relaxation via a common mechanism involving a covalent DNA-(3′-phosphotyrosyl)-protein intermediate. Genes encoding topoisomerase I activities have been identified from over a dozen cellular sources. The encoded proteins vary in size from 765 to 1019 amino acids. In addition, viral topoisomerase I genes have been cloned form five different genera of vertebrate poxviruses including vaccinia virus, Shope fibroma virus, Orf virus, fowlpox virus and at least one insect poxvirus. The poxvirus topoisomerases are fairly uniform in size (314-333 amino acids), and, like the eukaryotic topo I enzyme, carry an active site tyrosine residue located near the carboxy terminus within the conserved active site sequence Ser-Lys-X-X-Tyr. The poxvirus DNA topoisomerases further show approximately 35% amino acid identity (see e.g. Shuman (1998) Biochimica et Biophysica Acta 1400: 321-37).

Vaccinia virus topoisomerase is a 314 amino acid eukaryotic type I topoisomerase which binds and cleaves duplex DNA at the specific target sequence 5′-(T/C)CCTT-3′. Cleavage occurs by a transesterification reaction in which the CCCTTp↓N phosphodiester is attacked by the active site tyrosine (Tyr-274) resulting in the formation of a DNA-(3′-phosphotyrosyl) protein adduct. Cleavage can occur with small CCCTT-containing oligonucleotides as long as there are at least six nucleotides upstream and two nucleotides downstream of the scissile phosphate (Shuman (1991) J Biol Chem 266: 11372-79). The 30 covalently bound topoisomerase catalyzes a variety of DNA strand transfer reactions. It can either religate the CCCTT-containing strand across the same bond originally cleaved (as occurs during the relaxation of supercoiled DNA) or it can ligate the strand to a heterologous acceptor DNA 5′ end, thereby creating a recombinant nucleic acid molecule. Notably, a virtually irreversible or “suicide” cleavage occurs when the CCCTT-containing substrate contains no more than fifteen base pairs 3′ of the scissile bond, because the short leaving strand dissociates from the protein-DNA complex. In enzyme excess, more than 90% of the suicide substrate is cleaved. The suicide intermediate can transfer the incised CCCTT strand to DNA acceptor which corresponds to either a 5′ end of the DNA suicide substrate (intramolecular religation) complementary strand, to yield a hairpin structure, or to a second nucleic acid with a free 5′-OH, to yield an intermolecular ligation product. Intermolecular religation requires an exogenous 5′-OH terminated acceptor strand, the sequence of which is complementary to the single strand tail of the noncleaved strand in the immediate vicinity of the scissile phosphate. In the absence of an acceptor strand, the topoisomerase can transfer the CCCTT strand to water, releasing a 3′-phosphate-terminated hydrolysis product, or to glycerol, releasing a 3′-phosphoglycerol derivative. Indeed a vaccinia topoisomerase I-activated DNA intermediate can be religated to the 5′-OH end of an RNA molecule, thereby allowing rapid formation of DNA-RNA covalent adducts (see WO 98/56943). Furthermore, vaccinia topoisomerase activates DNA-RNA substrates as long as RNA segments are limited to regions downstream of the scissile phosphate (Shuman (1998) Molecular Cell 1: 741-48). Accordingly, the invention can be applied to the coupling of adaptors to RNA molecules with a free 5′-OH moiety.

Although preferred embodiments of the invention make use of vaccinia virus topoisomerase I and oligonucleotide adaptors carrying the sequence CCCTT or TCCTT, the invention anticipates that other topoisomerase I activities and alternative topoisomerase recognition sequences may be used in conjunction with the invention. For example, activation of the adaptor may be effected by active mutant derivatives of vaccinia topoisomerase (see e.g. Cheng et al. (1997) J Biol Chem 272: 8263-69) or even by an amino terminal deletion mutant of vaccinia topoisomerase which lacks the amino-terminal 80 amino acids (Cheng et al. (1998) 273: -11589-95). Furthermore, still other topoisomerase I-encoding sequences have been cloned, as discussed above, and their recognition sequences may be readily elucidated using methods known in the art (see Shuman (1998) Biochimica et Biophysica Acta 1400: 321-37). In addition, vaccinia topoisomerase I, or another topoisomerase activity, can be mutated randomly or in a directed manner so as to alter its DNA recognition specificity subtly or dramatically. Standard methods of random and site-directed mutagenesis are known in the art (see e.g. Sambrook et al. (1989) Molecular Cloning: A Laboratory Manual Cold Spring Harbor Press, §§ 15.1-15.113). Standard and automated high-throughput screening methods allow the rapid characterization of a large number of mutant topoisomerase I activities for retention of wild-type activity or specific alterations in sequence recognition and specificity.

The invention provides for the creation of topoisomerase I-activated adaptor sequences by a variety of methods. In general, activation occurs by incubating a target adaptor sequence which includes a topoisomerase recognition/cleavage sequence. Exemplary conditions for activation are known in the art and can be found in U.S. Pat. No. 5,766,891, the contents of which are incorporated by reference herein.

4.4. Nucleic Acids

The invention provides target nucleic acids, homologs thereof, and portions thereof. Preferred nucleic acids have a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, and more preferably 85% homologous and more preferably 90% and more preferably 95% and even more preferably at least 99% homologous with a nucleotide sequence of a specified gene or gene fragment or target nucleic acid sequence. Nucleic acids at least 90%, more preferably 95%, and most preferably at least about 98-99% identical with a nucleic sequence or complement thereof are of course also within the scope of the invention. In preferred embodiments, the nucleic acid is mammalian and in particularly preferred embodiments, includes all or a portion of the nucleotide sequence corresponding to the coding region of a target gene such as a cDNA molecule of the target gene sequence.

The invention also pertains to isolated nucleic acids comprising a nucleotide sequence encoding target polypeptides, variants and/or equivalents of such nucleic acids. The term equivalent is understood to include nucleotide sequences encoding functionally equivalent target polypeptides or functionally equivalent peptides having an activity of a specific target protein. Equivalent nucleotide sequences will include sequences that differ by one or more nucleotide substitution, addition or deletion, such as allelic variants; and will, therefore, include sequences that differ from the nucleotide sequence of the target gene due to the degeneracy of the genetic code.

Preferred nucleic acids are vertebrate cDNA nucleic acids. Particularly preferred vertebrate cDNA nucleic acids are mammalian. Regardless of species, particularly preferred vertebrate cDNA nucleic acids encode polypeptides that are at least 60%, 65%, 70%, 72%, 74%, 76%, 78%, 80%, 90%, or 95% similar or identical to an amino acid sequence of a vertebrate protein. In one embodiment, the nucleic acid is a cDNA encoding a polypeptide having at least one bio-activity of the subject polypeptide.

Still other preferred nucleic acids of the present invention encode a target polypeptide which is comprised of at least 2, 5, 10, 25, 50, 100, 150 or 200 amino acid residues. For example, such nucleic acids can comprise about 50, 60, 70, 80, 90, or 100 base pairs. Also within the scope of the invention are nucleic acid molecules for use as probes/primer or antisense molecules (i.e. noncoding nucleic acid molecules), which can comprise at least about 6, 12, 20, 30, 50, 60, 70, 80, 90 or 100 base pairs in length.

Another aspect of the invention provides a nucleic acid which hybridizes under stringent conditions to a specified nucleic acid. Appropriate stringency conditions which promote DNA hybridization, for example, 6.0× sodium chloride/sodium citrate (SSC) at about 45 [symbol] C, followed by a wash of 2.0× SSC at 50 [symbol] C, are known to those skilled in the art or can be found in Current Protocols in Molecular Biology, John Wiley & Sons, N.Y. (1989), 6.3.1-6.3.6 or in Molecular Cloning: A Laboratory Manual, Cold Spring Harbor Press (1989). For example, the salt concentration in the wash step can be selected from a low stringency of about 2.0× SSC at 50 [symbol] C to a high stringency of about 0.2× SSC at 50 [symbol] C. In addition, the temperature in the wash step can be increased from low stringency conditions at room temperature, about 22 [symbol] C, to high stringency conditions at about 65 [symbol] C. Both temperature and salt may be varied, or temperature and salt concentration may be held constant while the other variable is changed. In a preferred embodiment, a nucleic acid of the present invention will bind to a vertebrate cDNA nucleic acid sequence or complement thereof under moderately stringent conditions, for example at about 2.0× SSC and about 40 [symbol] C.

Nucleic acids having a sequence that differs from a specified nucleotide sequences or complement thereof due to degeneracy in the genetic code are also within the scope of the invention. Such nucleic acids encode functionally equivalent peptides (i.e., peptides having a biological activity of a target polypeptide) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of the polypeptide. However, it is expected that DNA sequence polymorphisms that do lead to changes in the amino acid sequences of the subject polypeptides will exist among mammals. One skilled in the art will appreciate that these variations in one or more nucleotides (e.g., up to about 3-5% of the nucleotides) of the nucleic acids encoding polypeptides having an activity of a target polypeptide may exist among individuals of a given species due to natural allelic variation.

### 5. EXAMPLES

5.1. Topoisomerase-mediated Cloning of a T7 Promoter onto a cDNA

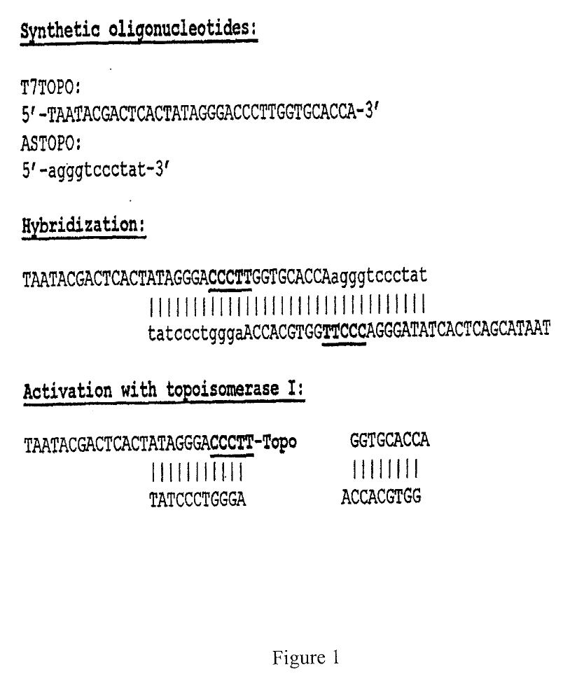

Standard adaptors may be designed for any particular application. In this example, we prepared universal adaptors for incorporation of a T7 RNA polymerase promoter onto a PCR product. The adaptor preparation starts by hybridization of two synthetic oligonucleotides. As shown in FIG. 1, the sequence of the first oligonucleotide is 5′-TAATACGACTCACTATAGGGACCCTTGGTGCACCA-3 (T7TOPO; SEQ ID NO. 1)′; and the sequence of the second oligonucleotide is 5′-AGGGTCCCTAT-3′ (ASTOPO; SEQ ID NO. 2). The structure of the oligonucleotides allows them to hybridize with formation of two topoisomerase I recognition sites within one hybrid. DNA hybrids were created by combining equimolar amounts of the T7TOPO and the ASTOPO oligonucleotides at 65 [symbol] C, followed by slow cooling of the mixture to 25 [symbol] C at a rate of about 0.5 [symbol] C/minute. Hybridization forms a stable complex of oligonucleotides with two recognition sites within the DNA duplex (FIG. 1). The existence of two nicks in the double strand hybrid does not affect the ability of the topoisomerase activity to recognize, cleave and form a covalent activated intermediate with the T7TOPO oligonucleotide strand (FIG. 1). This complex was found to be stable for weeks when stored in 50% glycerol at −20 [symbol] C.

Adaptor activation was performed by incubation of 8 pmol hybrid DNA with 5 units of vaccinia virus topoisomerase I (Epicentre) at 37 [symbol] C for 15 minutes. Next, PCR products generated from genomic DNA and single-stranded cDNA were generated as target nucleic acids for incorporation of a T7 promoter sequences using the topoisomerase activated adaptors. Two oligonucleotides, corresponding to sense and antisense sequences of the human PRL-1 gene were used to amplify a 483 bp fragment of the gene from human genomic DNA. The PRL-1 gene encodes a protein tyrosine phosphatase present in regenerating liver which is also expressed in foveal cells of the human retina. The sense oligonucleotide corresponded to positions 10021-10041 of the PRL-1 gene and had the sequence GAAGCACATGTCTTTAATGTC (SEQ ID NO. 3), while the antisense oligonucleotide corresponded to positions 100503-100481 of the PRL-1 gene and had the sequence GAACTAACATTAATACACATCAC (SEQ ID NO. 4). Based on the sequences of human red and green cone pigment cDNAs, sense (GTACCACCTCACCAGTGTCT, SEQ ID NO. 5) and antisense (AAATGATGGCCAGAGACCA, SEQ ID NO. 6) primers, corresponding to positions 156-176 and 443-423 of the red/green cone pigment cDNA respectively, were used to generate a 288 bp PCR product from monkey oligo(dT)-primed first strand cDNA.

Three microliters of each unpurified PCR product was incubated with toposiomerase activated adaptors for 5 minutes at room temperature. The adaptor carrying the T7 promoter, and the process for forming the activated adaptors is shown in FIG. 1. The reaction of topoisomerase activated adaptors with acceptor DNA is apparently complete within five minutes at room temperature and, typically, purification of acceptor DNA prior to reaction is not required. The modified acceptor DNA may be amplified by PCR with primers specific to the target cDNA sequence.

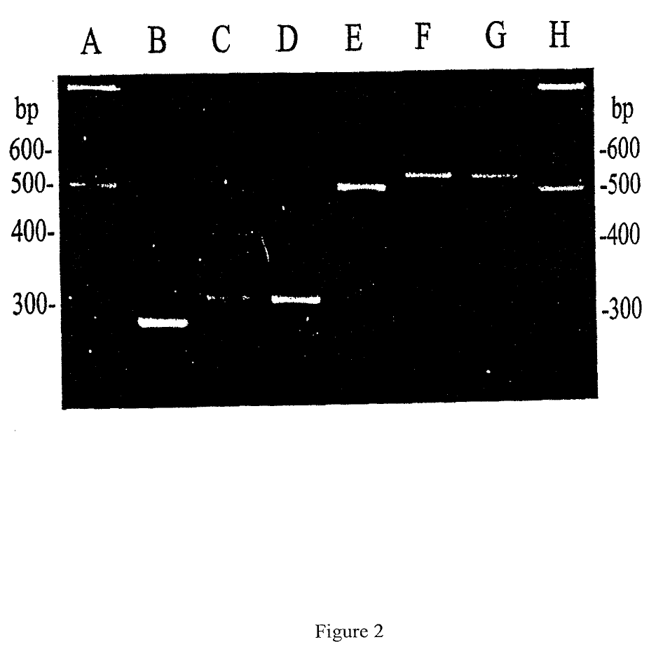

Incorporation of the T7 promoter sequence into the PCR products was confirmed by successful amplification of, and increased molecular weight of, the final PCR products visualized on a high resolution agarose gel (FIG. 2). FIG. 2 shows original PCR products as well as recombinant PCR products which have been re-amplified with sense and antisence primers coupled with T7 primer and separated on 3% SFR-agarose (lanes A and H are 100 bp size markers; lane B is the 288 bp fragment of red/green pigment cDNA; lane C is the fragment of red/green pigment cDNA with incorporated T7 promoter re-amplified with sense and T7 primers; lane D is the same as lane C after re-amplification with antisense and T7 primers; lane E is the 483 bp amplification product fragment of the PRL-1 gene; lane F is the fragment of the PRL-1 gene with T7 promoter, re-amplified with sense and T7 primers; and lane G is the same as lane F, but re-amplified with antisense primers).

For additional proof, the purified PCR products with T7 promoters were sequences using T7 or gene-specific primers. Sequencing confirmed the identity of the PCR products as T7 promoter-linked human PRL-1 gene and red/green cone pigment cDNA sequences respectively.

5.2. Generation of Labeled Probes for in situ Hybridization with Topo-activated Templates

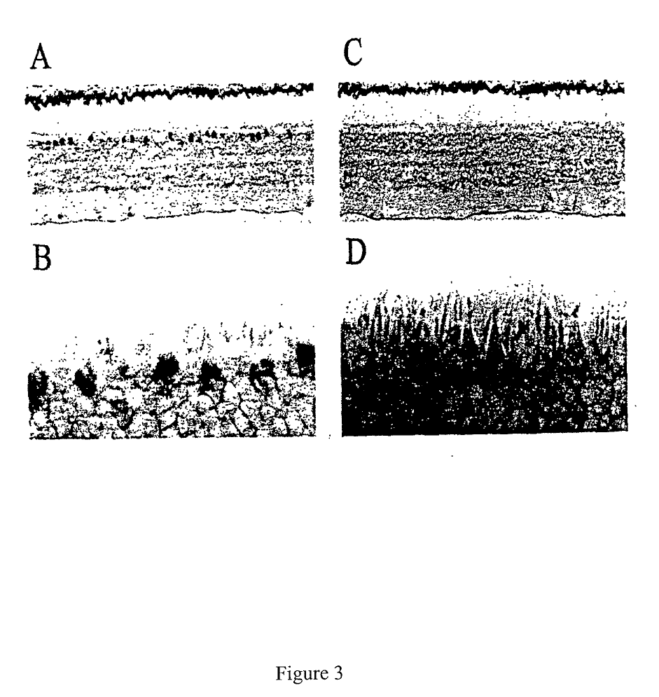

As an example of an application of the above-described approach, fragments of red/green cDNAs with incorporated T7 promoter sequences were used to produce cRNA probes by in vitro transcription with phage T7 RNA polymerase. The RNA probes were labeled with digoxigenin by incorporation of DIG-11-UTP during synthesis. The yields of reactions were 2-5 micrograms of DIG-labeled RNA as estimated by dot blotting with anti-DIG antibodies conjugated with alkaline phosphatase against control DIG-labeled RNA. Separate in vitro transcription reactions were run for sense and antisense probes for red/green pigment cDNA. Cryosections of monkey retina were hybridized with antisense and sense probes for red/green pigment cDNA. The bound probe was detected by incubation with anti-DIG antibodies conjugated with alkaline-phosphatase, followed by color staining using NBT/BCIP reagents. Distinct staining of cones was observed in the sections hybrized with antisense probes, while sense probes gave no signal (FIG. 3).

FIG. 3 shows the in situ hybridization signals obtained with monkey retinal tissue samples using the cRNA probes for red/green cone pigments. Monkey retina 7 mm cryosections were hybridized with antisense (panels A and B) or sense (panels C and D) cRNA probes which were transcribed in vitro from PCR products with T7 promoters. Magnification on micrographs A and C is 50×, and on micrographs B and D is 250×.

5.3. Design of Adaptor Sequences

The method provides a general means for incorporating useful sequences from an oligonucleotide into a target (acceptor) DNA sequence. Using this approach, commercially available topoisomerase activated adaptors may be developed which would provide a time- and cost-efficient means of incorporating nucleic acid sequences which provide any of a number of functions to a target nucleic acid sequence. For example a phage T7, T3 or SP6 RNA polymerase promoter or particular “sticky ends” or any other modification may incorporated into an acceptor/target DNA molecule such as a PCR product, a linearized plasmid or a restriction fragment. Also, this approach may be used to label the ends of an acceptor/target DNA with oligonucleotides containing modified residues (e.g. biotinylated, FITC or digoxigenin conjugated, etc.)

The longer oligonucleotide may be adapted to carry any useful sequence such as an RNA polymerase promoter sequence at the 5′-end in addition to a recognition site for vaccinia virus topoisomerase I (CCCTT) within 10 bases of the 3′ end (underlined sequence in FIG. 1). The 3′-end oligonucleotide also performs two other functions—i.e., it forms duplex DNA downstream of the recognition site and defines specificity for acceptor DNA which has either blunt ends (e.g. PCR products generated with proofreading DNA polymerase) or 3′ A overhangs (e.g. PCR products generated with Taq DNA polymerase). The shorter oligonucleotide should be designed to be complementary to the longer one at the toposiomerase I recognition sequence (i.e. 5′-AGGG-3′, which is complementary to 5′-CCCT-3′ of the T7TOPO oligonucleotide) as well as an additional few nucleotides of upstream sequence (i.e. 5′-TCCCTAT′3′, which is complementary to 5′-ATAGGGA-3′ in the T7TOPO oligonucleotide). Upon hybridization the oligonucleotides form double-stranded DNA upstream and at the topoisomerase recognition site. Moreover, if the oligonucleotides are designed for acceptor DNA with 3′ A overhangs, it should be shorter by one base providing complementarity to the first four bases of the recognition site. Topoisomerase I cleaves the DNA at the recognition site forming a covalent bond with the 3′-phosphate at the incised strand. Heterologous acceptor DNA may be covalently bound through the 3′-end phosphodiester bond instead of the cleaved fragment if the following requirements are met: the acceptor DNA is longer than 12 base pairs, the acceptor DNA has 3′-A overhangs, and the acceptor DNA has 5′-dephosphorylated ends.

Additional considerations in adaptor design include the possibility that the acceptor or target DNA molecule contains a CCCTT topoisomerase recognition sequence within 10 base pairs of a 3′ end. In such a case it is possible that topoisomerase carried over from the activation or released from the activated oligonucleotide adaptor may subsequently attack and cleave the acceptor/target molecule. The carryover of unreacted topoisomerase may potentially be prevented by purification of activated adaptors or by using saturating concentration of hybridized complex (i.e. adaptor oligoes in molar excess of the concentration of topoisomerase enzyme). The effect of topoisomerase released during reaction of the activated adaptors may be overcome by developing optimal conditions for the reaction using standard methodologies.

5.4. Vaccinia Virus Topoisomerase I

Vectors for the expression of vaccinia virus topoisomerase I may be generated using standard cloning methods (see e.g. Sambrook et al. (1989) Molecular Cloning: A Laboratory Manual Cold Spring Harbor Press). The amino acid sequence of vaccinia topoisomerase I (SEQ ID No. 8) and the nucleic acid sequence which encodes it (SEQ ID No. 7; GenBank Accession No. LI 3447) are shown below.

Vaccinia Topoisomerase I Protein Sequence:

MRALFYKDGKLFTDNNFLNPVSDDNPAYEVLQHVKIPTHLTDVVVYEQTWEEALTRLIF
VGSDSKGRRQYFYGKMHVQNRNAKRDRIFVRVYNVMKRINCFINKNIKKSSTDSNYQL
AVFMLMETMFFIRFGKMKYLKENETVGLLTLKNKHIEISPDEIVIKFVGKDKVSHEFVVH
KSNRLYKPLLKLTDDSSPEEFLFNKLSERKVYECIKQFGIRIKDLRTYGVNYTFLYNFWT
NVKSISPLPSPKKLIALTIKQTAEVVGHTPSISKRAYMATTILEMVKDKNFLDVVSKTTFD
EFLSIVVDHVKSSTDG

Vaccinia Topoisomerase I Gene, Nucleotide Sequence:

ATGCGTGCACTTTTTTATAAAGATGGTAAACTCTTTACCGATAATAATTTTTTAAATC
CTGTATCAGACGATAATCCAGCGTATGAGGTTTTGCAACATGTTAAAATTCCTACTC
ATTTAACAGATGTAGTAGTATATGAACAAACGTGGGAGGAGGCGTTAACTAGATTA
ATTTTTGTGGGAAGTGATTCAAAAGGACGTAGACAATACTTTTACGGAAAAATGCAT
GTACAGAATCGCAACGCTAAAAGAGATCGTATTTTTGTTAGAGTATATAACGTTATG
AAACGAATTAATTGTTTTATAAACAAAAATATAAAGAAATCGTCCACAGATTCCAAT
TATCAGTTGGCGGTTTTTATGTTAATGGAAACTATGTTTTTTATTAGATTTGGTAAAA
TGAAATATCTTAAGGAGAATGAAACAGTAGGGTTATTAACACTAAAAAATAAACAC
ATAGAAATAAGTCCCGATGAAATAGTTATCAAGTTTGTAGGAAAGGACAAAGTTTC
ACATGAATTTGTTGTTCATAAGTCTAATAGACTATATAAGCCGCTATTGAAACTGAC
GGATGATTCTAGTCCCGAAGAATTTCTGTTCAACAAACTAAGTGAACGAAAGGTATA
TGAATGTATCAAACAGTTTGGTATTAGAATCAAGGATCTCCGAACGTATGGAGTCAA
TTATACGTTTTTATATAATTTTTGGACAAATGTAAAGTCCATATCTCCTCTTCCATCA
CCAAAAAAGTTAATAGCGTTAACTATCAAACAAACTGCTGAAGTGGTAGGTCATAC
TCCATCAATTTCAAAAAGAGCTTATATGGCAACGACTATTTTAGAAATGGTAAAGGA
TAAAAATTTTTTAGATGTAGTATCTAAAACTACGTTCGATGAATTCCTATCTATAGTC
GTAGATCACGTTAAATCATCTACGGATGGATGA

5.5. Polymerase Chain Reaction Amplification

Polymerase chain reactions (PCR) utilize primer extension primers in a pairwise array as is well known. In general, to conduct a PCR reaction on a DNA sequence, one selects the desired PCR primer pair, and determines for each primer, the 3′ primer and the 5′ primer, which oligonucleotides of preselected sequence to produce, using the present methods. Thereafter, one admixes the prepared oligonucleotide compositions with a target for PCR amplification to form a PCR reaction admixture, ready for the PCR reaction. Certain permutations on PCR reaction methodologies will readily be apparent to one skilled in the art. PCR amplification methods are described in detail in U.S. Pat. Nos. 4,683,192, 4,683,202, 4,800,159, and 4,965,188, and at least in several texts including “PCR Technology: Principles and Applications for DNA Amplification”, H. Erlich, ed., Stockton Press, New York (1989); and “PCR Protocols: A Guide to Methods and Applications”, Innis et al., eds., Academic Press, San Diego, Calif. (1990).

The PCR reaction is performed by mixing the PCR primer pair, preferably a predetermined amount thereof, with the template nucleic acid having the sequence to be amplified, preferably a predetermined amount thereof, in a PCR buffer to form a PCR reaction admixture. The admixture is maintained under polynucleotide synthesizing conditions for a time period, which is typically predetermined, sufficient for the formation of a PCR reaction product, thereby producing an amplified PCR reaction product.

The PCR reaction is performed using any suitable method. Generally it occurs in a buffered aqueous solution, i.e., a PCR buffer, preferably at a pH of 7-9, most preferably about 8. Preferably, a molar excess (for genomic nucleic acid, usually about 10⁶:1 primer:template) of the primer is admixed to the buffer containing the template strand. A large molar excess is preferred to improve the efficiency of the process.

The PCR buffer also contains the deoxyribonucleotide triphosphates DATP, dCTP, dGTP, and dTTP and a polymerase, typically thermostable, all in adequate amounts for primer extension (polynucleotide synthesis) reaction. The resulting solution (PCR admixture) is heated to about 90° C.-100° C. for about 1 to 10 minutes, preferably from 1 to 5 minutes. After this heating period the solution is allowed to cool to 35° to 60° C., and preferably 40° to 500 C. depending upon the actual base composition as is known, which is preferable for primer hybridization. The synthesis reaction may occur at from room temperature up to a temperature above which the polymerase (inducing agent) no longer functions efficiently. Thus, for example, if DNA polymerase is used as inducing agent, the temperature is generally no greater than about 40° C. An exemplary PCR buffer comprises the following: 50 mM KCl; 10 mM Tris-HCl; pH 8.3; 1.5 mM MgCl2; 0.001% (wt/vol) gelatin, 200 mu M dATP; 200 mu M dTTP; 200 mu M dCTP; 200 mu M dGTP; and 2.5 units Thermus aquaticus DNA polymerase I (U.S. Pat. No. 4,889,818) per 100 microliters of buffer.

The amplifying polymerase may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′ end of each primer and proceed in the direction of 5′ to 3′ along the template strand, until synthesis terminates, producing molecules of different lengths. There may be inducing agents, however, which initiate synthesis at the 5′ end and proceed in the above direction, using the same process as described above.

The polymerase also may be a compound or system which will function to accomplish the synthesis of RNA primer extension products, including enzymes. In preferred embodiments, the inducing agent may be a DNA-dependent RNA polymerase such as T7 RNA polymerase, T3 RNA polymerase or SP6 RNA polymerase. These polymerases produce a complementary RNA polynucleotide. The high turn over rate of the RNA polymerase amplifies the starting polynucleotide as has been described by Chamberlin et al., The Enzymes, ed. P. Boyer, PP. 87-108, Academic Press, New York (1982). Another advantage of T7 RNA polymerase is that mutations can be introduced into the polynucleotide synthesis by replacing a portion of cDNA with one or more mutagenic oligodeoxynucleotides (polynucleotides) and transcribing the partially-mismatched template directly as has been previously described by Joyce et al., Nucleic Acid Research, 17:711-722 (1989). Amplification systems based on transcription have been described by Gingeras et al., in PCR Protocols, A Guide to Methods and Applications, pp 245-252, Academic Press, Inc., San Diego, Calif. (1990).

If the inducing agent is a DNA-dependent RNA polymerase and therefore incorporates ribonucleotide triphosphates, sufficient amounts of ATP, CTP, GTP and UTP are admixed to the primer extension reaction admixture and the resulting solution is treated as described above.

PCR is typically carried out by thermocycling i.e., repeatedly increasing and decreasing the temperature of a PCR reaction admixture within a temperature range whose lower limit is about 10° C. to about 40° C. and whose upper limit is about 90° C. to about 100° C. The increasing and decreasing can be continuous, but is preferably phasic with time periods of relative temperature stability at each of temperatures favoring polynucleotide synthesis, denaturation and hybridization.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

We claim:

1. A nucleic acid with a 5′ end and a 3′ end comprising a first functional nucleotide sequence and a scissile strand topoisomerase I cleavage motif sequence, wherein the scissile strand topoisomerase I cleavage motif sequence is located 3′ to the first functional nucleotide sequence and provides a scissile strand topoisomerase I cleavage site that is not more than 10 bases from the 3′ end of the nucleic acid.

2. The nucleic acid of claim 1, wherein the scissile strand topoisomerase cleavage motif sequence is selected from the group consisting of: CCCTT and TCCTT.

3. The nucleic acid of claim 1, wherein the first functional nucleotide sequence is selected from the group consisting of: a prokaryotic promoter sequence, a eukaryotic promoter sequence, a viral promoter sequence, a mutational sequence, a polypeptide tag encoding sequence, a nucleic acid tag sequence, a terminator sequence, a fusible protein encoding sequence, a radioactively labeled nucleotide sequence, a chemically labeled nucleotide sequence and an intronic sequence.

4. An adaptor comprising a first nucleic acid with a 5′ end and a 3′ end and comprising a scissile strand topoisomerase I cleavage motif having a 5′ motif sequence contiguous with a 3′ motif terminal nucleotide, said 3′ motif terminal nucleotide being contiguous with a palindromic sequence of not less than two nucleotides nor more than 10 nucleotides and said palindromic sequence being contiguous with a 3′ end nucleotide that is complementary to the 3′ motif terminal nucleotide of the scissile strand topoisomerase I cleavage motif.

5. The adaptor of claim 4 further comprising a second nucleic acid having a 5′ end sequence that is complementary to the 5′ motif sequence of the scissile strand topoisomerase I cleavage motif.

6. The first nucleic acid of the adaptor of claim 4, wherein the 3′ motif terminal nucleotide of the scissile strand topoisomerase I cleavage motif is T and the 5′ motif sequence of the scissile strand topoisomerase I cleavage motif is selected from the group consisting of CCCT and TCCT.

7. The first nucleic acid of the adaptor of claim 4 further comprising a restriction endonuclease site located 5′ to the scissile strand topoisomerase I cleavage motif.

8. The first nucleic acid of the adaptor of claim 4 further comprising a 5′ end sequence that is complementary to the 5′-overhang of a restriction endonuclease site.

9. The first nucleic acid of claim 7 or claim 8, wherein the restriction endonuclease is selected from the group consisting of: BamH I, Bgl II, Cla I, Dde I, Eae I, Eag I, EcoR I, Hind III, Kas I, Mbo I, Mlu I, Nco I, Nde I, Nhe I, Not I, PaeR7 I, Sal I, Sau3A, Spe I, Sty I, Xba I, Xho I and Xma I.

10. The first nucleic acid of the adaptor of claim 4, further comprising a first functional nucleotide sequence selected from the group consisting of: a prokaryotic promoter sequence, a eukaryotic promoter sequence, a viral promoter sequence, a mutational sequence, a polypeptide tag encoding sequence, a nucleic acid tag sequence, a terminator sequence, a fusible protein encoding sequence, a radioactively labeled nucleotide sequence, a chemically labeled nucleotide sequence and an intronic sequence.

11. A method for joining an adaptor sequence to a target nucleic acid sequence comprising:
providing a nucleic acid adaptor of claim 5,
providing a target nucleic acid with a one base 3′ overhang nucleotide that is complementary to the 3′ motif terminal nucleotide of the scissile strand topoisomerase cleavage motif, and
incubating the nucleic acid adaptor with the target nucleic acid in the presence of a topoisomerase I activity,
thereby joining the adaptor sequence to the target nucleic acid sequence.

12. The method of claim 11, wherein the first nucleic acid of the adaptor of claim 5 further comprises a functional nucleotide sequence that is 5′ to the scissile strand topoisomerase I cleavage motif.

13. The method of claim 12, wherein the functional nucleotide sequence is selected from the group consisting of: a prokaryotic promoter sequence, a eukaryotic promoter sequence, a viral promoter sequence, a mutational sequence, a polypeptide tag encoding sequence, a nucleic acid tag sequence, a terminator sequence, a fusible protein encoding sequence, a radioactively labeled nucleotide sequence, an intronic sequence.

14. The method of claim 12, wherein the functional nucleotide sequence is a phage promoter selected from the group consisting of: an SP6 promoter, a T3 promoter and a T7 promoter.

15. The method of claim 11, further comprising the step of amplifying the joined product.

16. The method of claim 15, wherein the joined product is amplified by a polymerase chain reaction utilizing a first primer specific to the nucleic acid adaptor and a second primer specific to the target nucleic acid sequence.

17. The method of claim 11, wherein the target nucleic acid is generated by a polymerase chain reaction of a target genomic or a target cDNA sequence with a 5′ sense strand primer and a 3′ anti-sense strand primer.

18. The method of claim 17, wherein the adaptor provides a functional nucleotide sequence that is a promoter sequence and further comprising the steps of preparing at least two separate amplification reactions from the joined product comprising:
a first amplification reaction with 3′ anti-sense strand primer and a first adaptor primer; and
a second amplification reaction with a 5′ sense strand primer and a second adaptor primer, wherein the first adaptor primer comprises a sequence in the first nucleic acid of the adaptor and the second adaptor primer comprises a sequence in the second nucleic acid of the adaptor.

19. The method of claim 18 further comprising the step of isolating the product of either the first amplification reaction or the second amplification reaction.

20. The method of claim 19 further comprising contacting the amplification product with an RNA polymerase activity which recognizes said promoter sequence.


SEQUENCE LISTING (12 sequences)

SEQ ID NO: 1 - DNA, 35 bp, Artificial Sequence
  Description of Artificial Sequence Synthetic oligonucleotide
taatacgactcactatagggacccttggtgcacca

SEQ ID NO: 2 - DNA, 11 bp, Artificial Sequence
  Description of Artificial Sequence Synthetic oligonucleotide
agggtccctat

SEQ ID NO: 3 - DNA, 21 bp, Artificial Sequence
  Description of Artificial Sequence Synthetic oligonucleotide
gaagcacatgtctttaatgtc

SEQ ID NO: 4 - DNA, 23 bp, Artificial Sequence
  Description of Artificial Sequence Synthetic oligonucleotide
gaactaacattaatacacatcac

SEQ ID NO: 5 - DNA, 20 bp, Artificial Sequence
  Description of Artificial Sequence Primer
gtaccacctcaccagtgtct

SEQ ID NO: 6 - DNA, 19 bp, Artificial Sequence
  Description of Artificial Sequence Primer
aaatgatggccagagacca

SEQ ID NO: 7 - DNA, 945 bp, Vaccinia virus
atgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataagccgctattgaaactgacggatgattctagtcccgaagaatttctgttcaacaaactaagtgaacgaaaggtatatgaatgtatcaaacagtttggtattagaatcaaggatctccgaacgtatggagtcaattatacgtttttatataatttttggacaaatgtaaagtccatatctcctcttccatcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatga

SEQ ID NO: 8 - protein, 314 aa, Vaccinia virus
MRALFYKDGKLFTDNNFLNPVSDDNPAYEVLQHVKIPTHLTDVVVYEQTWEEALTRLIFVGSDSKGRRQYFYGKMHVQNRNAKRDRIFVRVYNVMKRINCFINKNIKKSSTDSNYQLAVFMLMETMFFIRFGKMKYLKENETVGLLTLKNKHIEISPDEIVIKFVGKDKVSHEFVVHKSNRLYKPLLKLTDDSSPEEFLFNKLSERKVYECIKQFGIRIKDLRTYGVNYTFLYNFWTNVKSISPLPSPKKLIALTIKQTAEVVGHTPSISKRAYMATTILEMVKDKNFLDVVSKTTFDEFLSIVVDHVKSSTDG

SEQ ID NO: 9 - DNA, 17 bp, Artificial Sequence
  Description of Artificial Sequence T7 phage promoter
taatacgactcactata

SEQ ID NO: 10 - DNA, 24 bp, Artificial Sequence
  Description of Artificial Sequence T3 phage promoter
ttattaaccctcactaaagggaag

SEQ ID NO: 11 - DNA, 23 bp, Artificial Sequence
  Description of Artificial Sequence SP6 phage promoter
atttaggtgacactatagaatac

SEQ ID NO: 12 - DNA, 46 bp, Artificial Sequence
  Description of Artificial Sequence Synthetic oligonucleotide
taatacgactcactatagggacccttggtgcaccaagggtccctat